(19) United States
(12) Patent Application Publication
(10) Pub. No.: US 2025/0332198 A1
(43) Pub. Date: Oct. 30, 2025

(54) TARGETED ELIMINATION OF SENESCENT CELLS BY GAMMA-DELTA T CELLS

(71) Applicant: SENS RESEARCH FOUNDATION, Mountain View, CA (US)

(72) Inventors: Amit SHARMA, Mountain View, CA (US); Teshahun Dessale ADMASU, Mountain View, CA (US)

(73) Assignee: SENS RESEARCH FOUNDATION, Mountain View, CA (US)

(21) Appl. No.: 18/871,067

(22) PCT Filed: Jun. 1, 2023
(86) PCT No.: PCT/US2023/024187

§ 371 Date: Dec. 2, 2024

Related U.S. Application Data
(60) Provisional application No. 63/347,914, filed on Jun. 1, 2022.

(51) Int. Cl. A61K 35/17; A61K 31/352; A61K 31/506; A61K 31/635; A61K 31/675; A61K 40/11; A61K 40/31; A61P 43/00

(52) U.S. Cl. CPC A61K 35/17; A61K 31/352; A61K 31/506; A61K 31/635; A61K 31/675; A61K 40/11; A61K 40/31; A61P 43/00

(57) ABSTRACT

Senescent cells are implicated in aspects of age-related decline in health and may contribute to certain diseases. Embodiments include compositions and methods for the treating senescence-associated diseases and disorders by the administering gamma delta (γδ) T cells. The gamma delta T cells target senescent cells over non-senescent cells. The methods can also be used for slowing the aging process and/or reducing signs of aging.

### FIELD OF THE INVENTION

The invention relates to therapeutics, and more specifically, it relates to therapeutic methods using gamma-delta T cells for treating senescence-associated diseases and disorders.

### BACKGROUND

Aging can be defined as the process of becoming older. In humans, aging represents the accumulation of changes over time and can encompass physical, psychological, and social changes. Advanced age is the greatest risk factor for many chronic diseases. More than 90% of adults aged 65 or older experience at least one chronic disease such as cancer, diabetes, or cardiovascular disease. Aging phenotypes and pathologies, including diverse age-associated diseases and disorders, are causally linked to the accumulation of senescent cell burden with age.

Senescent cells are characterized by irreversible cell-cycle arrest of proliferation-competent cells, morphological and metabolic changes, altered gene expression, chromatin reorganization, and a unique pro-inflammatory senescence-associated secretory phenotype (SASP). Replicative senescence is activated upon serial passage of cells in culture (or as cells become older in an organism). Senescence can also be induced by range of different insults that include oncogene activation, irradiation and exposure to chemotherapeutic drugs. Moreover, there are several drugs (e.g., CDK4/CDK6 inhibitors such as Palbociclib) that induce senescence.

Senescent cells in older adults are associated with many diseases (e.g., cancer and fibrosis) and contribute to chronic inflammation and damage to surrounding tissues. Senescent cells can become resistant to apoptosis and have up-regulation of anti-apoptotic pathways which defend them against their own inflammatory senescence-associated secretory phenotype (SASP). This allows senescent cells to survive despite killing neighboring cells. It has been demonstrated that removal of senescent cells via genetic manipulation in transgenic mouse models can prevent or delay tissue dysfunction, improve age-related pathologies, and extend health span. This suggests that removal of senescent cell burden in aging adults, merits further study as a therapeutic target of interest for the treatment and prevention of disease of aging.

Efforts to develop therapies for senescent related ailments have focused on methods of eliminating senescent cells without affecting non-senescent cells. Several senolytic compounds have shown promising results in mice and human cell culture models. Known compounds include dasatinib and quercetin, piperlongumine and Bcl2-family inhibitors such as ABT263 and ABT737. While these agents have demonstrated some success in selectively targeting senescent cells, they have limitations. Due to the significant side-effects, they cannot be administered at doses effective to achieve a desired effect. For example, ABT263 (also known as navitoclax) has dose-limiting platelet toxicity which presents the risk of causing thrombocytopenia. Navitoclax, fisetin and dasatinib plus quercetin (D+Q) are reported to destroy macrophages. Accordingly, there is a need to identify new methods and compounds with senolytic properties.

Recent studies have begun to elucidate the immune system's ability to recognize senescent cells and target them for removal/destruction. Innate, or nonspecific immunity refers to the defense system which protects one against all antigens. Innate immunity involves barriers that keep harmful materials from entering one's body. These barriers form the first line of defense in the immune response. In contrast, the adaptive immune system (i.e., the acquired immune system) is a subsystem of the immune system that includes specialized systemic cells and processes to eliminate pathogens or prevent their growth.

Natural Killer (NK) cells appear to be the most important cell type involved in this process, although neutrophils and abT cells have also been reported to selectively kill senescent cells (SC), with perhaps some involvement of macrophages. In addition to conventional αβ T cells, other subclasses of T cells include mucosal-associated invariant T cells (MAIT), invariant Natural Killer T cells (INKT), germline-encoded mycolyl lipid-reactive T cells (GEMT), and γδT cells. Peripheral blood αβ-T cells make up approximately 50% of lymphocytes whereas a smaller proportion of CD3+ cells (˜0.5-10%) are γδ-T cells. The γδT cells recognize their target antigens irrespective of MHC haplotype, and mediate anti-tumor response without causing graft versus host disease (GvHD). They exert cytotoxic activity via the granzyme-perforin axis or via antibody dependent cellular cytotoxicity (ADCC). γδT cells can also release cytokines such as TNF-α and IFN-γ. Moreover, these cells can also phagocytose tumor antigens and apoptotic or live cancer cells (possibly through the scavenger receptor CD36) and induce maturation of dendritic cells by increasing TNF-α production. Furthermore, γδT cells can interact with B cells to promote immunoglobulin class switching and cross-present antigens to CD8+ T cells.

In humans, gamma delta (γδ) T cells constitute a minor subset among T lymphocytes, constituting 1%-10% of mature circulating T cells. Unlike the majority of αβ T cells, most γδT cells (>70%) are CD4−CD8−, some (˜30%) are CD8+CD4− and very few (<1%) are CD4+CD8−. Gamma delta T cells are a subset of T cells that provide a link between innate and acquired immune responses. This cell undergoes V-(D)-J segment rearrangement to produce antigen-specific γδT cell receptors (γδ TCR) and γδT cells. γδT cells represent a small proportion of the total T cell population in mammals that are approximately 1-5% of T cells in peripheral blood and lymphoid organs and are predominantly expressed in epithelial rich compartments such as skin, liver, digestive tract, respiratory and reproductive tract. Unlike αβ TCRs that recognize antigens bound to the major histocompatibility complex (MHC), γδ TCR is a key component of bacterial antigens, viral antigens, stress antigens expressed by affected cells, and intact proteins or non-peptide compounds.

There is a need for improved methods to identify senescent cells and target them for apoptosis. The present invention includes the use of gamma delta T cells to selectively target senescent cells with high specificity and reliability. Also included are methods treating age-related diseases and conditions by selective elimination of senescent cells.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

The invention relates to gamma delta T cells and therapeutic uses thereof along with methods of generating (i.e., isolating/enriching) gamma delta T cells.

Methods are described for using gamma delta (γδ) T cells and therapeutic products to target senescent cells (SCs). The methods can be used to treat senescence associated diseases or disorders. The methods can also be used to slow the aging process and/or reduce signs of aging. In embodiments, the gamma delta T cells are modified by, for example, T-cell receptor (TCR) gene transfer or chimeric antigen receptor (CAR) expression.

Accordingly, embodiments include methods for selectively inducing apoptosis of senescent cells to treat a senescence-associated disease or disorder using gamma delta T cells. Embodiments also include methods for selectively inducing apoptosis of senescent cells to slow the aging process and/or reduce signs of aging.

Embodiments also include therapies for senescence-associated diseases and disorders using gamma delta (γδ) T cells. In aspects, the therapies can delay tissue dysfunction, improve age-related pathologies and/or extend health span.

In embodiments, the methods described herein include pharmaceutical formulations containing therapeutic agents that selectively kill senescent cells (i.e., selectively kills senescent cells over non-senescent cells or compared with non-senescent cells). The therapeutic agents can include gamma delta (γδ) T cells. In embodiments, the gamma delta (γδ) T cells are enriched and/or modified.

Embodiments also include a method of treating an ailment (i.e., a senescence-associated disease or disorder) or slowing the aging process/reducing signs of aging. The method can include steps of (a) enriching gamma delta (γδ) T cells and (b) administering the gamma delta (γδ) T cells therapeutically. The method can also include a step of modifying the gamma delta (γδ) T cells. In aspects, the γδT cells are sourced from the subject. In aspects, the γδT cells are sourced from one or more donors (e.g., from PBMCs).

Embodiments also include methods of removing senescent cells for diagnostic and/or therapeutic purposes.

Embodiments also include methods of combination therapy as gamma delta (γδ) T cells can be administered in combination with know senolytic or senomorphic drugs. Accordingly, embodiments include methods of treating a senescence-associated disease or disorder that includes administering gamma delta (γδ) T cells with one or more senolytic agents (e.g., a small molecule) to selectively kill senescent cells over non-senescent cells. The senolytic agent can be, for example, dasatinib, quercetin, fisetin or navitoclax.

Embodiments also include methods of identifying senescent cells for targeted therapy. Embodiments also include methods of removing senescent cells from an affected tissue of a subject.

Embodiments include methods of decreasing senescent cell burden. Embodiments also include methods of treating, reducing the likelihood of occurrence of, or delaying onset of a senescent cell-associated disease or disorder. A formulation containing gamma delta T cells can be administered to a subject intravenously.

Embodiments also include methods of generating gamma delta T cells and augmenting and/or enhancing their function(s). In aspects, the methods of isolating and/or enriching gamma delta T cells include supplementing cells with interleukin-2 (IL-2) and zoledronate (ZOL).

One aspect is a method of isolating and/or enriching gamma delta T cells (γδ) T cells. The method can include steps of: (a) culturing a population of cells comprising γδT cells with a phosphoantigen to expand the γδT cells; (b) culturing the expanded γδT cells with artificial antigen-presenting cells expressing a Fc receptor, and an anti-CD3 antibody and (c) modifying the γδT cells to express CAR, wherein the CAR comprises an extracellular antigen-binding domain of NKG2D. In aspects, the Fc receptor is CD64. In aspects, the phosphoantigen is zoledronic acid. In aspects, the population of cells are peripheral blood mononuclear cells (PBMCs).

In aspects, the isolated/enriched gamma delta T cells are administered to a subject to treat a senescence associated disease or disorder. In aspects, the isolated/enriched gamma delta T cells are administered to a subject to reduce the number of senescent cells and/or reduce signs of aging.

In aspects, γδT cells can be genetically edited to improve therapeutic potential. Such genetic editing may be performed by any means known in the art, such as, for example, by the use of artificial nuclease(s). Such genetic editing may redirect the specificity of the γδT cells through the expression of a chimeric antigen receptor (CAR) or T-cell receptor (TCR). Such genetic editing may improve the potency of the γδT cells by improving homing, cytokine production, recycle killing, and/or improved engraftment.

In one embodiment, a pharmaceutical composition is provided comprising a cell composition of the present embodiments and a pharmaceutically acceptable carrier.

In one embodiment, a method of treating a disease in a patient is provided that includes administering an effective amount of a cell composition or a pharmaceutical composition as described herein. In aspects, the disease is related to aging.

In one embodiment, a method of treating a disease in a patient is provided comprising producing a cell composition according to the methods of the present embodiments and administering an effective amount of said cell composition to a patient in need thereof.

In some aspects, methods are provided for treating an individual with a medical condition comprising the step of providing an effective amount of cells from the population of cells described herein, including more than once in some aspects, such as at least 1, 2, 3, 4, 5, 6, 7, 8, 9, 10, 11, 12, 13, 14, or more days apart.

In one embodiment, a composition comprising a cell population or pharmaceutical composition of the present embodiments is provided for use in the treatment of a senescence associated ailment in a patient. In one aspect, the cell composition may be allogeneic to the patient. In another aspect, the cell composition may be autologous to the patient. In another embodiment, the use of a cell population of the present embodiments in the manufacture of a medicament for the treatment of a disease is provided.

### BRIEF DESCRIPTION OF THE DRAWINGS

The accompanying drawings illustrate aspects of the present invention. In such drawings:

FIG. 1A is a flow chart of steps of a protocol for isolating and enriching Vγ9Vδ2 T cells. Total PBMC were treated with 5 μM of zoledronic acid and 500iu ril-2 for 24 hours and incubated with ril-2 afterwards for ten days.

FIG. 1B is a FACS analysis that shows enrichment in the γδ TCR expressing cells among CD3 expressing T cells (i.e., CD3+ cells from total PBMC).

FIG. 1C is a FACS analysis that also shows enrichment in the γδ TCR expressing cells among CD3 expressing T cells (CD3+ and gdTCR+ cells).

FIG. 1E is a FACS analysis that shows percent of gamma delta T cells from total PBMC before and after enrichment from multiple donors. Most (i.e., 86%) of CD3+ cells also express Vδ2 TCR whereas none of CD3+ cells express Vδ1 TCR.

FIG. 1F is a graph that shows the percent of gamma delta T cells before (left) and after (right) enrichment.

FIG. 1G is a FACS analysis shows that about 85% the cells were double positive for γδ TCR and CD56.

FIG. 1H is a FACS analysis that shows no significant amount of g9Vd1+ cells after enrichment.

FIG. 2A is a graphical depiction of coculture optimization using the xCellegence® RTCA system which shows cell viability over time. Percent cytotoxicity of senescent and non-senescent IMR90 fibroblasts treated with different E:T ratio of GD T cells.

FIG. 2B is a bar chart showing the percentage of cytotoxicity of senescent (S) and non-senescent (NS) cells over time. Percent cytotoxicity of senescent and non-senescent IMR90 fibroblasts 24 hours after treatment.

FIG. 2C is a graph that shows the percentage cytolysis compared to the relative efficacy of enriched Vδ2 T cells toward senescent and non-senescent IMR90 target cells. Cytotoxicity of enriched GD T cells from multiple donors towards senescent and non-senescent IMR90 fibroblasts.

FIG. 2D shows cytotoxicity of enriched gamma delta T cells from multiple donors towards senescent and non-senescent human primary endothelial cells.

FIG. 2E shows the cytotoxicity of total PBMC towards senescent and non-senescent IMR90 fibroblasts.

FIG. 3A is a FACS analysis that identifies 5% CD3− cytotoxic cells (CD56+).

FIG. 3B is a FACS analysis that identifies CD3+gsTCR− cytotoxic cells (CD56+).

FIG. 3C is a bar graph that shows depletion of gdTCR− cells significantly reduced ab T cells in multiple donors. Percent of ab TCR+ cells from fresh PBMC, enriched cells and enriched followed by GD T cells selection.

FIG. 3D is a bar graph that shows depletion of gdTCR− cells significantly increased proportion of GD T cells in multiple donors.

FIG. 3E is a series of images that shows gdTCR+ and abTCR+ T cells population from fresh PBMC (C-i), enriched cells (C-ii) and enriched followed by GD T cells selection (C-iii).

FIG. 3F is a series of images that shows the same results from a different donor.

FIG. 4A is a graph that shows NKG2D neutralizing antibody significantly reduced cytotoxicity of gamma delta T cells.

FIG. 4B are immunofluorescent images that show senescent cells express CD277 (BTN3A) on their surface.

FIG. 4C is a graph that shows gdTCR neutralizing antibody significantly reduced cytotoxicity of gamma delta T cells.

FIG. 4D is a graph that shows combinations of NKG2D and gdTCR neutralizing antibody significantly reduced cytotoxicity of gamma delta T cells.

FIG. 4E is a graph that shows mevastatin treated senescent cells are

resistant to gamma delta T cells.

FIG. 5A shows cell viability determined by 7-AAD staining (GDT cells population in freshly isolated PBMC from multiple donors).

FIG. 5B shows a single suspension based on forward scatter.

FIG. 5C shows a side scatter.

FIG. 5D shows GD T cells (CD3+gdTCR+) for multiple donors.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “senescence” refers to gradual deterioration of functional characteristics in living organisms. Cellular senescence is often defined as a stress-induced, durable cell cycle arrest of previously replication-competent cells. The effects of senescent cells can be thought of as beneficial or detrimental with regard to host physiology and disease, although in some contexts, senescent cells affect a disease state in a complex manner both promoting and opposing certain conditions.

As described herein, a senescent cell can be, for example, a senescent fibroblast, a senescent pre-adipocyte, a senescent epithelial cell, a senescent chondrocyte, a senescent neuron, a senescent smooth muscle cell, a senescent mesenchymal cell, a senescent macrophage or a senescent endothelial cell.

The term “senescence-associated disease or disorder” refers to an ailment that is associated with age and can include, for example, atherosclerosis, osteoarthritis, osteoporosis, hypertension, arthritis, cataracts, cancer, Alzheimer's disease, chronic obstructive pulmonary disease (COPD) and idiopathic pulmonary fibrosis. Other ailments (including age-related conditions) associated with age or senescence include hair graying, sarcopenia, adiposity, neurogenesis, fibrosis and glaucoma.

Still other ailments associated with age or senescence include cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke). A senescence-associated disease or disorder can also be an inflammatory or autoimmune disease or disorder (e.g., osteoarthritis, osteoporosis, oral mucositis, inflammatory bowel disease or kyphosis). A senescence-associated disease or disorder can also be a neurodegenerative disease (e.g., Alzheimer's disease, Parkinson's disease, Huntington's disease, dementia, mild cognitive impairment or motor neuron dysfunction). A senescence-associated disease or disorder can also be a metabolic disease (e.g., diabetes, diabetic ulcer, metabolic syndrome or obesity). A senescence-associated disease or disorder can also be a pulmonary disease (e.g., pulmonary fibrosis, chronic obstructive pulmonary disease, asthma, cystic fibrosis, emphysema, bronchiectasis or age-related loss of pulmonary function). A senescence-associated disease or disorder can also be an eye disease or disorder (e.g., macular degeneration, glaucoma, cataracts, presbyopia or vision loss). A senescence-associated disease or disorder is an age-related disorder can also be renal disease, renal failure, frailty, hearing loss, muscle fatigue, skin conditions, skin wound healing, liver fibrosis, pancreatic fibrosis, oral submucosa fibrosis or sarcopenia. A senescence-associated disease or disorder can also be a dermatological disease or disorder (e.g., eczema, psoriasis, hyperpigmentation, nevi, rashes, atopic dermatitis, urticaria, diseases or disorders related to photosensitivity or photoaging).

The term “senescence-associated ß-galactosidase,” “SA-β-gal” or “SABG” is a hypothetical hydrolase enzyme that catalyzes the hydrolysis of β-galactosides into monosaccharides only in senescent cells. Senescence-associated beta-galactosidase, along with p16Ink4A, can be used as a biomarker of cellular senescence.

The term “senolytic” or “senolytic agent” refers to a therapeutic such as a small molecule that can selectively or preferentially induce death of senescent cells. A senolytic agent may kill senescent cells by inducing (i.e., activating, stimulating or removing inhibition of) an apoptotic pathway that leads to cell death. Senolytic agents may be useful for treatment of senescence-associated diseases or disorders. For example, the drugs dasatinib, quercetin, fisetin and navitoclax have potential senolytic activities.

The term “biomarker” refers generally to a DNA, RNA, protein, carbohydrate, or glycolipid-based molecular marker, the expression or presence of which in a sample can be detected by standard methods (or methods disclosed herein) and is predictive or prognostic of the effective responsiveness or sensitivity of a mammalians subject with an ailment. Biomarkers may be present in a test sample but absent in a control sample, absent in a test sample but present in a control sample, or the amount or of biomarker can differ between a test sample and a control sample. For example, protein biomarkers can be present in such a sample, but not in a control sample, or certain biomarkers are seropositive in the sample, but seronegative in a control sample. Also, expression of such a biomarker may be determined to be higher than that observed from a control sample. The terms “marker” and “biomarker” are used herein interchangeably.

The amount of the biomarker can be measured in a test sample and compared to the “normal control level,” utilizing techniques such as reference limits, discrimination limits, or risk defining thresholds to define cutoff points and abnormal values for an ailment. The normal control level means the level of one or more biomarkers or combined biomarker indices typically found in a subject not suffering from the ailment. Such normal control level and cutoff points can vary based on whether a biomarker is used alone or in a formula combining with other biomarkers into an index. Alternatively, the normal control level can be a database of biomarker patterns from previously tested subjects who did not experience the ailment over a clinically relevant time.

Tests to measure biomarkers and biomarker panels can be implemented on a variety of diagnostic test systems. Diagnostic test systems are apparatuses that typically include means for obtaining test results from biological samples. Examples of such means include modules that automate the testing (e.g., biochemical, immunological, nucleic acid detection assays). Some diagnostic test systems are designed to handle multiple biological samples and can be programmed to run the same or different tests on each sample. Diagnostic test systems typically include means for collecting, storing and/or tracking test results for each sample, usually in a data structure or database. Examples include well-known physical and electronic data storage devices (e.g., hard drives, flash memory, magnetic tape, paper printouts). It is also typical for diagnostic test systems to include means for reporting test results. Examples of reporting means include visible display, a link to a data structure or database, or a printer. The reporting means can be a data link to send test results to an external device, such as a data structure, data base, visual display, or printer.

The term “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

The term “treating” or “treatment” refers to one or more of (1) inhibiting the disease, condition or disorder in an individual who is experiencing or displaying the pathology or symptomatology of the disease, condition or disorder (i.e., arresting further development of the pathology and/or symptomatology); and (2) ameliorating the disease condition or disorder in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The compositions disclosed herein may be administered via any of the common routes, as long as it is able to reach a desired tissue, for example, inhaling, intraperitoneal, intravenous, intramuscular, subcutaneous, intradermal, oral, topical, intranasal, intrapulmonary, or intrarectal administration.

The term “subject” refers to those who a susceptible to an ailment (e.g., a disease related to senescence) or who are suspected of having or diagnosed with the ailment. However, any subject to be treated with the therapeutic methods described herein is included without limitation.

The term “T cell” refers to a type of lymphocyte. T cells are one of the important white blood cells of the immune system and play a central role in the adaptive immune response. T cells can be distinguished from other lymphocytes by the presence of a T-cell receptor (TCR) on their cell surface. There are three main types of T cells: cytotoxic, helper and regulatory. Each of them has a different role in the immune response.

T cells can also be classified as conventional adaptive T cells and innate-like T cells. Conventional adaptive T cells include helper CD4+ T cells, cytotoxic CD8+ T cells, memory T cells and regulatory CD4+ T cells. Innate-like T cells include natural killer T cell, mucosal associated invariant T cells and gamma delta T cells.

Cytotoxic T cells (also known as TC, killer T cell, or cytotoxic T-lymphocyte (CTL)) are a sub-group of T cells that induce the death of cells that are infected with viruses (and other pathogens) or are otherwise damaged or dysfunctional. Cytotoxic T cells have a co-receptor (i.e., CD8) on their cell surface. CD8 partners with the T cell receptor and with MHC class I molecules, which allows cytotoxic T cells to recognize normal cells that are infected by a pathogen. When the cytotoxic T cell recognizes the infected cell, it becomes activated and produces molecules that kill the infected cell, destroying the pathogen in the process.

CD4+ lymphocytes, also called “helper” T cells, are immune response mediators, and play an important role in establishing and maximizing the capabilities of the acquired immune response. Helper T cells have a different co-receptor (i.e., CD4) on their cell surface. CD4 also partners with the T cell receptor but interacts with MHC class Il molecules instead of MHC class I molecules. This allows helper T cells to recognize pathogen peptides that have been displayed by antigen presenting cells. When helper T cells recognize a peptide on an antigen presenting cell, they become activated and begin to produce molecules called cytokines that signal to other immune cells. Thus, these cells have no cytotoxic or phagocytic activity; and cannot kill infected cells or clear pathogens, but, in essence “manage” the immune response, by directing other cells to perform these tasks.

The term “unconventional T cells” include those lymphocytes that express aδTCR and may commonly reside in an epithelial environment such as the skin, gastrointestinal tract, or genitourinary tract. Their role is to recognize infections and cancer cells and regulate inflammatory responses that arise in these tissues. Unconventional T-cells include CD1-restricted T cells, MR1-restricted mucosal associated invariant T cells (MAIT cells), MHC class lb-reactive T cells, and γδT cells. These T cells can recognize lipids, small-molecule metabolites and specially modified peptides in contrast to major histocompatibility complex (MHC) reactive T cells.

Gamma delta T cells (γδT cells) are a unique T cell subpopulation that are rare in secondary lymphoid organs but enriched in many peripheral tissues, such as the skin, intestines and lungs. By rapidly producing large amounts of cytokines, γδT cells make key contributions to immune responses in these tissues. Gamma delta T cells possess an alternative T cell receptor (TCR) as opposed to CD4+ and CD8+ αβ T cells and share characteristics of helper T cells, cytotoxic T cells and natural killer cells. Like other “unconventional” T cell subsets bearing invariant TCRs, such as CD1d-restricted natural killer T cells, γδT cells exhibit characteristics that place them at the border between innate and acquired immunity. On one hand, γδT cells may be considered a component of adaptive immunity in that they rearrange TCR genes via V(D)J recombination, which also produces junctional diversity, and develop a memory phenotype. On the other hand, however, the various subsets may also be considered part of the innate immune system where a restricted TCR or NK receptors may be used as a pattern recognition receptor. Human gamma delta T cells can be activated by phospho-antigens and aminobisphosphonates such as zoledronate.

The dominant gamma delta T cell subtype in peripheral blood are Vγ9Vδ2 T cells which are found in humans and constitute 0.5-10% of lymphocytes in human blood. Most Vγ9Vδ2 T cells are double negative (DN) for the co-receptors CD4 and CD8; about 20-30% are single positive CD8 and 0.1-7% express CD4. The functional role of these co-receptors in the context of γδT cells remains unknown as Vγ9Vδ2 T cells recognize antigen in an HLA independent fashion.

Vγ9Vδ2 T cells respond in a T cell receptor (TCR)-dependent manner to phosphoantigens which are generated by a variety of microorganisms. Vγ9Vδ2 T cells have been proposed as cells capable of performing tumor cell elimination and intracellular defense against parasitic bacteria and parasites without being restricted by major histocompatibility antigens. Further, cancer treatment with use of Vγ9Vδ2 T cells obtained from human peripheral blood is currently under research.

The term “phosphoantigens” or “PAgs” refers to small molecules which are metabolites of the methyl erythritol phosphate pathway in microbial pathogens and the eukaryotic mevalonate (MVA) pathway in tumor cells. Phosphoantigens such as isopentenyl pyrophosphate (IPP) play a significant role in γδT cells mediated cytotoxicity towards pathogens and cancerous cells. IPP binding to the intracellular domain of BTN3A leads to a conformational change that alters the extracellular domain, enabling recognition by the γδTCR. PAgs stimulate Vγ9Vδ2 cells in the presence of antigen-presenting cells, suggesting a strict requirement for dedicated antigen-presenting molecules.

The term “Butyrophilin-3A” or “BTN3A” refers to a group of immunoglobulins present on the surface of different cell types, including innate and cancer cells. Recent studies have identified butyrophilin (BTN) 3A1 as the molecule necessary to stimulate Vγ9Vδ2 cells. Butyrophilin 3A (BTN3A) is upregulated in many types of cancer cells and has been widely investigated as a ligand that mediates the activation of γδT cells. The rate-limiting step in the mevalonate pathway is the transformation of HMGCoA to mevalonic acid via the HMGCR enzyme. HMGCR activity and downstream products in the mevalonate pathway can therefore be inhibited by statins.

The term “Lysosomal-associated membrane protein 1” or “LAMP-1” also known as lysosome-associated membrane glycoprotein 1 and CD107a (Cluster of Differentiation 107a), is a protein that in humans is encoded by the LAMP1 gene.

The term “Tripeptidyl-peptidase 1” or “TPP-1” also known as Lysosomal pepstatin-insensitive protease, is an enzyme that in humans is encoded by the TPP1 gene.

The term “antibody-dependent cellular cytotoxicity,” “ADCC” or “antibody-dependent cell-mediated cytotoxicity” refers to a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection.

The term “adoptive cell therapy,” “ACT” or “adoptive transplantation” refers to a treatment that uses a cancer patient's own T lymphocytes with anti-tumour activity, expanded in vitro and reinfused into the patient with cancer. This approach harvests naturally occurring T cells that have already infiltrated patients' tumors, and then activates and expands them. Then, large numbers of these activated T cells are re-infused into patients, where they can then seek out and destroy tumors.

The term “FC” or “flow cytometry” refers to a technique used to detect and measure physical and chemical characteristics of a population of cells or particles. The term “FACS” or “flow cytometry cell sorting” refers to a technique used to sort cells in which a particular cell type is separated from others contained in a sample on the basis of its physical or biological properties (e.g., size, morphological parameters, viability and protein expression). The homogeneous cell population obtained after sorting can be further studied.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” refers to a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### Detailed Description

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Different kinds of immune cells such as macrophages, NK cells, and T cells are known to remove senescent cells (SC). NK cells identify SC via NKG2D ligand such as MICA and ULBP2 and secrete perforin and granzyme for cytotoxic effector function in the elimination of SC. On the other hand, αβT cells recognize pathogens through the TCR receptor, which requires peptide antigen presentation to engage their cytotoxic activity. The major limitation of NK cells is that their effect can be impeded by inhibitory ligands such as NKG2A, while αβT cells are limited by the need for antigen presentation and the inhibitory ligand HLA-E. Gamma delta T cells (γδT cells) may overcome the limitations of NK cells and αβT cells because unlike NK cells they do not have a receptor for inhibitory legends and unlike αβT cells they do not require antigen presentation. Further, γδT cells have both adaptive and innate features that make them uniquely suited to coordinating an early response to the elimination of SC.

Gamma delta T cells (γδT cells) are T cells that express a unique T-cell receptor (TCR) composed of one γ-chain and one δ-chain. They are found in the gut mucosa, skin, lungs and uterus, and are involved in the initiation and propagation of immune responses. They are in relatively low abundance in the body. In humans, they are a minor subset among T lymphocytes and constitute 1%-10% of mature circulating T cells.

Unlike the majority of αβ T cells, most gamma delta T cells (i.e., more than 70%) are CD4−CD8−. Approximately 30% are CD8+CD4− and few (i.e., less than 1%) are CD4+CD8−. Like αβ T cells and B cells, the structural diversity of gamma delta T cells is dependent on V(D)J somatic recombination, which generates a set of highly diverse receptors for antigen recognition. However, the repertoire of gamma delta T cells is limited as compared to αβ T cells and B cells. This diversity is mainly generated in the complementary-determining region 3 (CDR3) of the TCR. Among various subtypes, the Vγ9Vδ2 T cells are about 50-95% of the peripheral gamma delta T cells in circulation. Applicants proposed the use of Vγ9Vδ2 T cells for therapeutic use, particularly for senolytic therapies.

Recent studies have reported that Vγ9Vδ2 T cells have potent and broad tumor cytotoxicity due to MHC-independency and recognition of targets based on phosphoantigens and MICA. They have relatively low propensity to secrete IL-17 and low sensitivity to immune inhibitory checkpoints like PD-1. Vγ9Vδ2 T cells are stimulated by prokaryotic isoprenoid pathway mevalonate isoprenoid pathway end products. The levels of these naturally occurring metabolites are too low to be detected as a dangerous signal by Vγ9Vδ2 T cells in normal cells whereas dysfunctional metabolism of malignant tumor cells can result in the accumulation of endogenous phosphoantigens.

As gamma delta T cells can function as innate immune cells, and there is overlap in several effector functions with NK cells, Applicants investigated whether gamma delta T cells are also involved in the immune surveillance of senescent cells. Applicants isolated and enriched gamma delta T cells from human peripheral blood mononuclear cells (PBMCs) and thereafter, demonstrated the efficacy of those cells for immune mediated clearance of senescent cells.

Applicants enriched gamma delta T cells from less than 5% to up to 85%. Further, flow cytometric analysis further revealed that Vγ9Vδ2 T cells were the predominant cells that were enriched. Further, upon coculture with senescent IMR-90 cells, Applicants observed that gamma delta T cells eliminated senescent cells in a dose dependent manner whereas the effect on non-senescent cells was negligible.

The results described below demonstrate that γδT cells can eliminate multiple types of SC. One of the major limitations of senolytic drugs is the nonspecific toxicity towards non-SC, attributable to their nonspecific inhibition of cell survival pathways and other metabolic pathways on which nonsenescent cells also rely. In contrast, the unique combinations of NKG2D and γδTCR on γδT cells allows for execution of specific cytotoxicity based on the presence of phosphoantigens and NKG2D ligands which are specific to cancerous, infectious, senescent, and other damaged cell types. The results also show that γδT cell enrichment and activation by zoledronate treatment of target cells renders them specifically cytotoxic to senescent cells (SC) rather than non-SC. Further, γδT cells will not have prolonged cytolytic effect as they have a short half-life. Taken together, the data herein demonstrates that specific activation of γδT cells can provide the basis of more specific approaches to eliminate SC, capitalizing on the benefits of SC destruction with less toxicity to normal cells.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof

### Methods

### Cell Culture

IMR-90 fibroblasts (ATCC, USA: Cat #CCL-186) were maintained at 37° C. in humidified air containing 5% CO₂ and 3% O₂. Fibroblasts were used at population doubling level (PDL) 30-47 and maintained in DMEM complete media containing Dulbecco's Modified Eagle's Medium (DMEM) (Corning; Cat #10-013-CV) supplemented with 10% Fetal Bovine Serum (FBS) (Millipore Sigma, USA; Cat #F4135) and 1× Penicillin-Streptomycin (Corning; Cat #30-001-CI). Cumulative PDL was calculated using the following equation:

$$PDL = \frac{log H - log S}{log 2}$$

where H is the number of cells at harvest and S is the number of cells seeded. Primary human endothelial cells purchased from Coriell Institute for medical research (AG10770) were maintained in promo cell basal medium MV2 (PromoCell; Cat #C-22221) supplemented with Growth Medium MV 2 Supplement Pack (PromoCell; Cat #C-39221) and assayed within less than a passage number of 10. Endothelial cells were maintained at 37° C. in humidified air containing 5% CO₂.

### Senescence Induction

Human IMR-90 fibroblasts were treated with 300 nM of doxorubicin hydrochloride (Millipore Sigma, USA; Cat #504042) in DMEM complete media for 24 hours and maintained in culture as described above.

### Gamma Delta (γδ) T Cell Isolation and Enrichment

The γδT cells were isolated and enriched from human blood as described in a published protocol using zoledronate and interleukin-2 (see, e.g., Kondo, M. et al. Expansion of human peripheral blood gamma delta T cells using zoledronate. J Vis Exp). The blood samples were obtained from healthy donors (n=5, age range 20-42) in heparin coated vacutainers. All subjects provided informed written consent. Inclusion criteria for healthy individuals included those who did not take medication that could impact immunity (e.g., corticosteroids) and had no clinical indication of immunodeficiency. Blood samples were diluted with 1× PBS (Corning; Cat #21-031-CV) and combined with Lymphocyte Separation Medium (Corning; Cat #25-072-CI) followed by density gradient centrifugation, according to the manufacturer's instructions. The aliquot of PBMC was analyzed for the proportion of the γδ T cells by flow cytometry. The remainder of PBMCs were cultured in RPMI complete media, 20% Fetal Bovine Serum (FBS) (Millipore Sigma, USA; Cat #F4135) with 500 IU/ml human rIL-2 (recombinant Interleukin-2) (Cat #TECIN teceleukin; Bulk Ro 23-6019) and 5 uM Zoledronic Acid (Zol) (Tocris, USA cat #6111) for 24hrs. The cells were expanded with rIL-2 for 10 days with fresh media changed every other day, before co-culturing with senescent or non-senescent IMR-90 fibroblasts.

### Flow Cytometry

Cells were resuspended in 100 μl of PBS. Cells were then incubated with APC-conjugated anti-human CD3 antibody (Miltenyi Biotec; Cat #130-113-135). FITC-conjugated anti-human gd TCR antibody (Miltenyi Biotec; Cat #130-113-503), PE-conjugated TCR Vδ1 Antibody (Miltenyi Biotec; Cat #130-120-440), FITC-conjugated TCR Vδ2 Antibody (Miltenyi Biotec; Cat #130-111-009), FITC-conjugated CD56 antibody (Miltenyi Biotec; Cat #130-113-312) and APC-conjugated CD56 antibody (Miltenyi Biotec; Cat #130-113-312) on ice for 30 minutes. Cells were washed with 1 ml of ice cold PBS and resuspended in 100 μl of ice cold PBS. Data were collected by flow cytometer (DB Accuri C6). Cell viability was determined by Propidium iodide (PI) staining and live cells were gated for downstream analysis. Data were analyzed using Flowlogic software (Miltenyi Biotech, Germany).

### Impedance Measurements

For measurement of background values, 50 μl of complete medium was added to E-Plates 96 (Agilent). Senescent and non-senescent cells were seeded in a total of 200 μl of medium per well at a density of 10,000 cells per well. gd T cells were added on top of senescent and non senescent cells at different effector to target (E:T) ratio. Cell viability was monitored using the RTCA MP (Agilent) instrument and the RTCA software (Agilent). Cells treated with 0.2% Triton X-100 was used as a 100% dead cell positive control for cytotoxicity assays. Cell index which reflects number of viable cells and cytotoxicity were recorded every 15 minutes. All experiments were performed in at least triplicates. To analyze the acquired data, CI values and percent cytotoxicity were exported.

### Senescence-Associated Beta-Galactosidase

SA-β Gal activity was detected using a commercial kit (Biovision®).

### Immunofluorescence

Cells were plated at a density of 10,000 cells per well using a 96 well plate and senescence was induced as documented above. On day 8 after senescence induction, non-senescent controls were added to the plate at 10,000 cells per well. 10 days after senescence induction, cells were fixed with 4% PFA for 15 minutes at room temperature. Cells were then permeabilized in 0.2% Triton X-100 for 15 minutes and blocked in 5% goat serum in PBS for 1 hour. Primary antibody was added and left overnight at 4° C. on a shaker. Primary antibodies used were gamma γH2AX (P Ser139 3F2 Novus) (1:1000 dilution) and Anti-HMGB1 antibody (ab18256 Abcam) (1:1000 dilution). CD277 immunofluorescence was performed on non-permeabilized cells using BTN3A1 Polyclonal antibody (Proteintech, 1:1000 dilution). After incubation with primary antibody, cells were incubated with fluorescent secondary antibody and Hoechst (Invitrogen H3570) (1:2000 dilution) for 1 hour at room temperature in the dark. Secondary antibodies used were Invitrogen Goat anti-Rabbit IgG (H+L) Cross-Adsorbed Secondary Antibody, Alexa Fluor 546 (Catalog #A-11010) and Goat anti-Mouse IgG (H+L) Highly Cross-Adsorbed Secondary Antibody, Alexa Fluor™ 488 (Catalog #A-11029). Images were acquired with an EVOS cell imaging system at 20× and 40× magnification. ImageJ software was used for image analysis.

### Western Blot

Cells were lysed in RIPA buffer (Cell Signaling technology, Catalog #9806S) with protease-phosphatase inhibitor cocktail (Cell Signaling technology, Catalog #5871). Cell suspensions were then incubated for 15 min on ice and sonicated for 30 seconds. The suspensions were cleared of debris by microcentrifugation at 4° C. for 15 min. Protein concentration was determined by BCA Protein Assay (Thermo Scientific, Catalog #23227). Electrophoresis was performed with 20 μg of protein per condition and transferred to PVDF membranes (Bio-Rad, Catalog #1620177) using a wet transfer apparatus (Invitrogen™ XCell II™ Blot Module). Membranes were incubated with primary antibody overnight at 4° C., followed by incubation with secondary antibody for 1 h at room temperature. Blots were developed using Azure Radiance Plus (SKU: AC2103) and visualized with an Azure 500 chemiluminescence imaging system.

### Gamma Delta (γδ) T Cell Isolation and Enrichment

The γδT cells were isolated and enriched from human blood using previously published protocol (Kondo et al, Cytotherapy 10, 842-856, 2008). The blood samples were obtained from healthy donors (n=5, age range 20-42) in heparin coated vacutainers. All subjects provided informed written consent. Inclusion criteria for healthy individuals included those who did not take medication that could impact immunity (e.g., corticosteroids) and had no clinical indication of immunodeficiency. Blood samples were diluted with 1× PBS and combined with Lymphocyte Separation Medium followed by density gradient centrifugation, according to the manufacturer's instructions. The aliquot of PBMC was analyzed for the proportion of the γδT cells by flow cytometry. The remainder of PBMCs were cultured in RPMI complete media, 20% Fetal Bovine Serum (FBS) with 500 IU/ml human rIL-2 (recombinant Interleukin-2) and 5 μM Zoledronic Acid (Zol) for 24 hours. The cells were expanded with rIL-2 for ten days with fresh media changed every other day, before co-culturing with senescent or non-senescent IMR-90 fibroblasts. After ten days of expansion, γδT cells were negatively selected using Miltenyi Biotec™ γδT cells isolation kit.

### Flow Cytometry

Cells were resuspended in 100 μl of PBS. Cells were then incubated with APC-conjugated anti-human CD3 antibody. FITC-conjugated anti-human γδ TCR antibody, PE-conjugated TCR Vδ1 Antibody, FITC-conjugated TCR Vδ2 Antibody, FITC-conjugated CD56 antibody and APC-conjugated CD56 antibody on ice for 30 minutes. Cells were washed with 1 ml of ice cold PBS and resuspended in 100 μl of ice cold PBS. Data were collected by flow cytometer (MACSQuant10, Miltenyi Biotec™). Cell viability was determined by PI staining and live cells were gated for downstream analysis. Data were analyzed using Flowlogic™ software.

### Real-Time Cytotoxicity Assay (xCELLigence®)

50 μL medium was added to E-Plates 96 for measurement of background values. SC and their respective non-senescent cells were seeded in an additional 100 μL medium at a density of 10,000 cells per well. Cell attachment was monitored using the RTCA MP instrument and the RTCA software until the plateau phase was reached, which was usually after approximately 24 hours. Floating cells were removed, and γδT cells were added on top of senescent and non-senescent cells at different E:T ratio. Cells treated with 0.2% Triton x-100 as a 100% dead cell positive control for cytotoxicity assay. Upon addition of gdT cells, impedance measurements were performed every 15 min for up to 36 hours. All experiments were performed at least in triplicate in three independent experiments for each donor and cell types. Changes in impedance were expressed as a cell index (CI) value, which derives from relative impedance changes corresponding to cellular coverage of the electrode sensors, normalized to baseline impedance values with medium only. Percent cytotoxicity was determined based on the relative CI value. To analyze the acquired data, CI values were exported, and percentage of lysis was calculated in relation to the control cells lacking any gdT cells.

### Quantification and Statistical Analysis

Statistical analysis was conducted using Graph Pad Prism 9. All data are presented as means±SEM. All cell culture datasets represent means of at least three experiments. Comparisons between groups were performed using a 2-tailed Student's t test, 1- or two-way ANOVA, as appropriate with appropriate correction for multiple comparisons. Statistical parameters can be found in figure legends.

### Example 1

### Enrichment of γδT Cells From Human PBMC

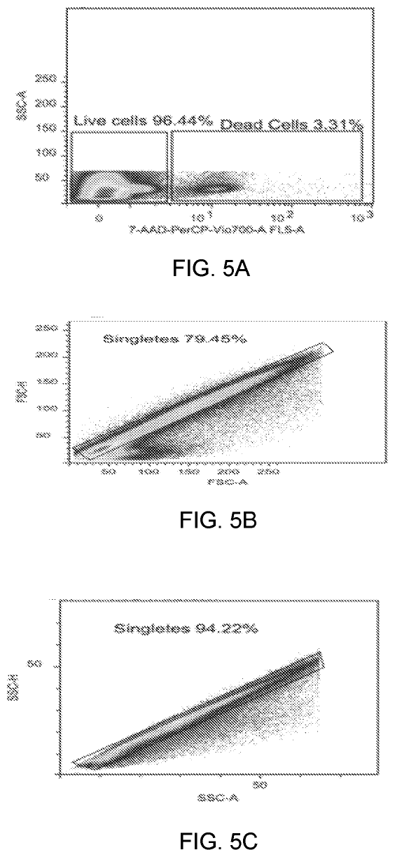

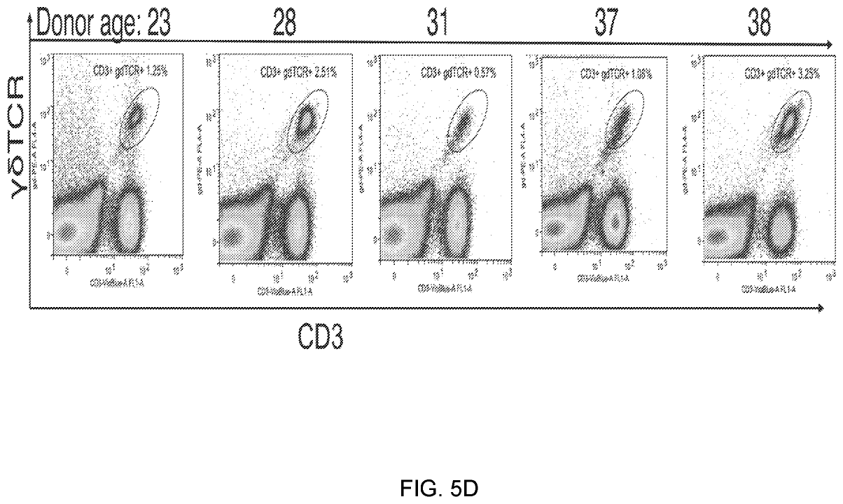

To determine the percentage of γδT cells in human PBMC, blood was collected from healthy donors (n=5, ages 20-42) and PBMCs were isolated. FACS analysis of human PBMCs indicated that less than 5% of total PBMCs was γδ TCR and CD3 positive (FIG. 5A). There are several classes of γδT cells, of which Vγ9Vδ2 T cells are the major one in PBMCs. We therefore determined the proportion of Vγ9Vδ2 T cells (hereafter “Vδ2”) in human PBMCs and found that about 3% of total PBMC were positive for Vγ9Vδ2 TCR (FIG. 5B). We did not observe any Vγ9Vδ1 expressing cells in the PBMCs (FIG. 5C and 5D).

Vδ2 T cells have been shown to have potent and broad tumor cytotoxicity and are less sensitive to immune inhibitory checkpoints such as PD-1 (see, e.g., Kunkele et al, Cells 9, 2020). As less than 5% of PBMCs are Vδ2 T cells, a previously published protocol was used to enrich Vδ2 T cells (Kondo et al., Cytotherapy, 10 842-856, 2008) to enable investigation of their effect on immunosurveillance of SC.

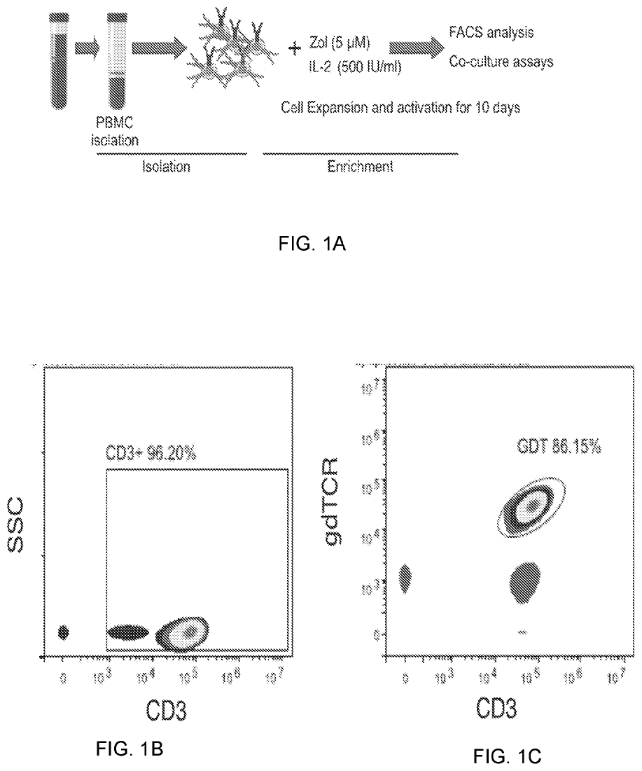

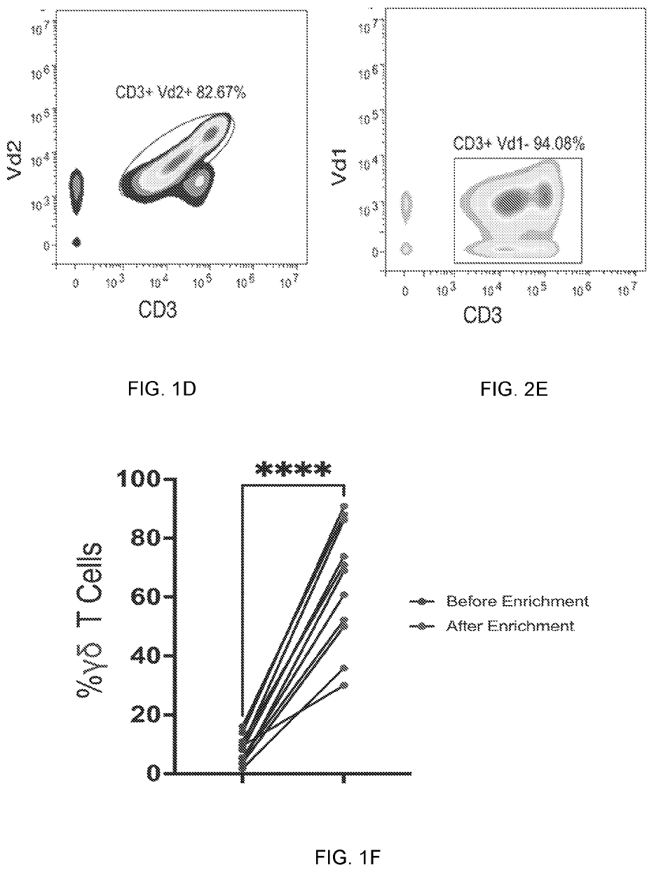

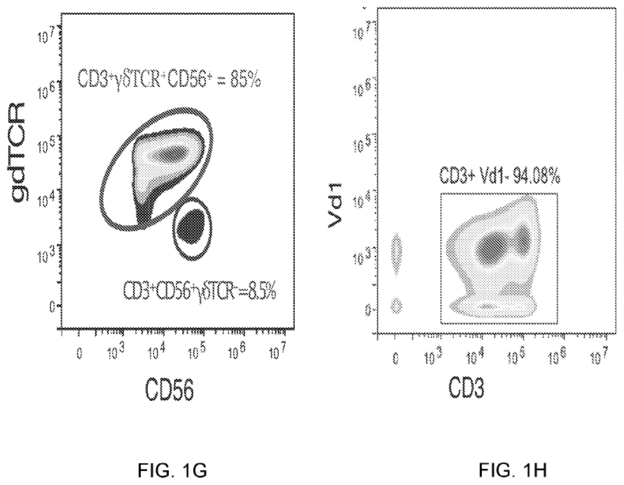

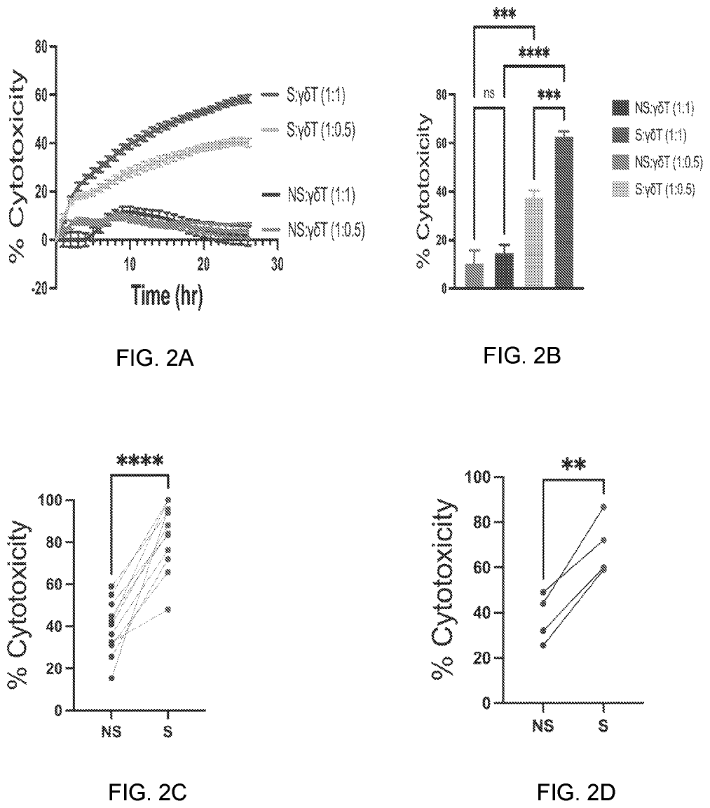

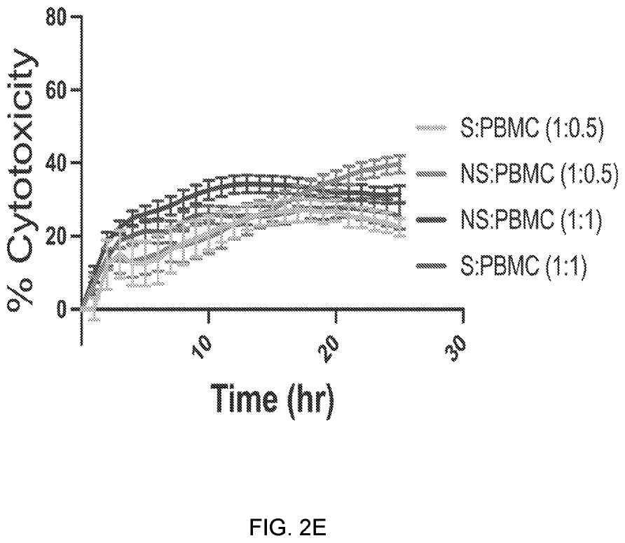

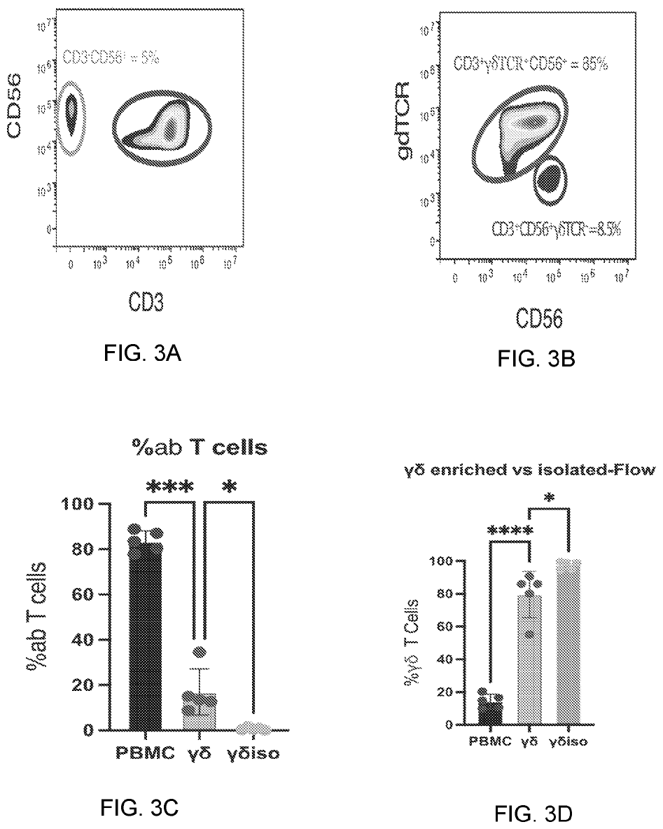

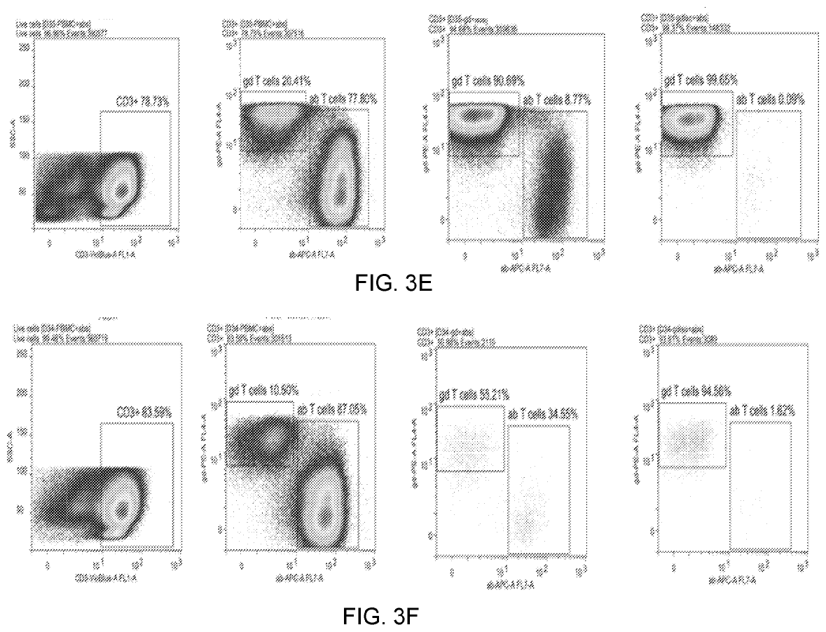

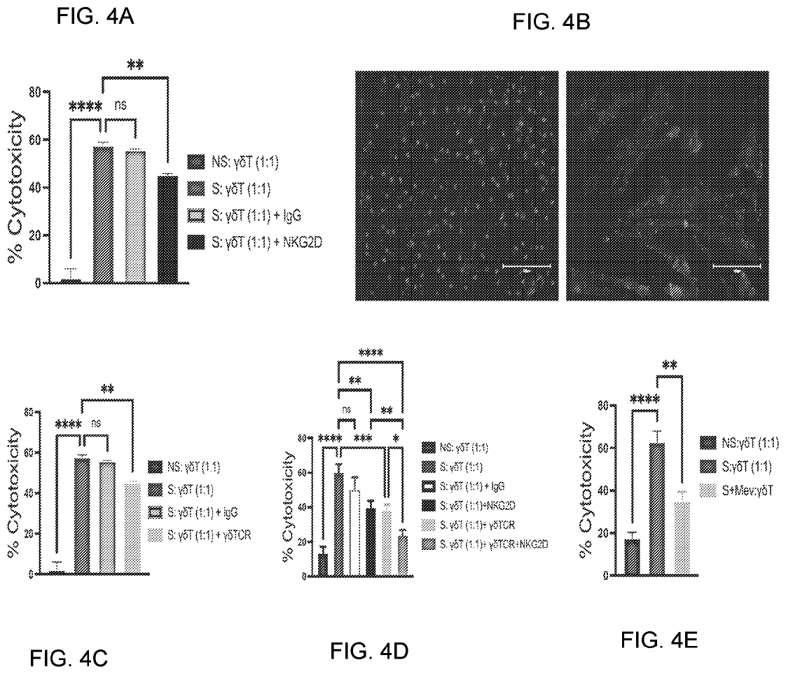

Human Vδ2 T cells respond to phosphoantigens such as isopentenyl pyrophosphate (IPP), which is produced in eukaryotic cells through the mevalonate pathway. IPP can be induced to accumulate in monocytes when PBMC are treated with aminobisphosphonates such as pamidronate or zoledronate that inhibit farnesyl pyrophosphate synthase (FPPS), an enzyme acting on IPP to produce downstream products in the mevalonate pathway. Monocytes efficiently taken up zoledronate and accumulate IPP, becoming antigen-presenting cells that stimulate Vδ2 T cells in the peripheral blood. A similar protocol was used which included culturing freshly isolated PBMCs from healthy donors with interleukin-2 (IL-2) and zoledronate (ZOL) in RPMI media for 24 hours and then maintaining the cells with only IL-2 for 10 days (FIG. 1A). A separate culture of PBMCs was also maintained with IL-2 only as a control. After 10 days of expansion, we ran flow analysis to determine the enrichment of γδT cells. The results showed a significant enrichment of γδ TCR and CD3-expressing T cells, from less than 10% in PBMC supplemented with IL-2 to 86% in PBMC treated with IL-2 and ZOL (FIGS. 2B and 2C). As expected, 86% of CD3+ cells also express Vγ9Vδ2 TCR and none of them express Vγ9Vδ1 TCR, thus showing that enriched cells are Vδ2 T cells (FIGS. 1E and 1F. We also showed that this protocol could be used to enrich γδ TCR expressing cells from samples isolated from multiple donors (FIG. 1E). Furthermore, flow cytometry analysis showed that about 85% the cells were double positive for γδ TCR and CD56 (FIG. 1F). As the expression of CD56 is often associated with the degree of activation, these data suggest that the protocol for γδT cells enrichment results in activation of these cells along with their enrichment.

### Example 2

### γδT Cells are Cytotoxic Towards Senescent Cells

To investigate if Vδ2 T cells enriched from human PBMCs can selectively kill SC, the doxorubicin model of senescence induction was used. Human fibroblasts (IMR90) were cultured and treated with doxorubicin to induce senescence; a robust senescence phenotype was confirmed using multiple makers. Untreated proliferating IMR90 cells were used as non-senescent controls. Separately, PBMCs from a healthy human donor were split into two cultures: one was maintained with IL-2 and the other was treated with ZOL to enrich Vδ2 T cells. Senescent or non-senescent IMR90 cells were seeded into wells, and PBMC or enriched Vδ2 T cells were added to these wells at different target-to-effector ratios. The xCELLigence® platform was used to measure cellular impedance as a continuous monitor of real-time kinetic behavior indicative of cell number and attachment. Triton-X was used as a positive control for cytotoxicity. Immune cells remain in suspension and do not adhere to the wells and as such contribute negligibly to impedance.

Using the xCELLigence® software, the percentage cytolysis was calculated and used for comparing the relative efficacy of enriched Vδ2 T cells toward senescent and non-senescent IMR90 target cells (FIG. 2A). Co-culture of PBMCs with senescent and non-senescent IMR90 cells did not show selective cytotoxicity towards SC. Similarly, co-culture of enriched Vδ2 T cells with non-senescent IMR90 cells showed minimal cell killing at two target: effector ratios of 0.5:1 and 1:1. In contrast, co-culturing similarly enriched Vδ2 T cells with senescent IMR90 cells induced a significant dose- and time-dependent increase in cytolysis of senescent fibroblasts (FIG. 2A). Moreover, even at the lowest target: effector (E:T) ratio of 0.5:1, enriched Vδ2 T cells were significantly more cytotoxic toward SC (FIG. 2A).

This finding was confirmed by repeating the experiment with PBMC or enriched Vδ2 T cells from multiple donors. The results showed that enriched Vδ2 T cells from multiple donors selectively killed SC. To determine if Vδ2 T cells kill SC from other cell types of origin, senescence was induced in primary human endothelial cells with doxorubicin treatment and repeated the co-culture assay. We found that Vδ2 T cells from multiple donors selectively killed senescent endothelial cells. These results indicate that enriched Vδ2 T cells can preferentially induce cytotoxicity of senescent over non-SC.

### Example 3

### Isolation of γδT Cells After Enrichment Increases the Specificity

Although Vδ2 T cells from multiple donors showed high cytotoxicity towards SC compared to non-SC, we observed that Vδ2 T cells enriched from some donors have relatively high cytotoxic towards non-SC as well. We were interested to know the source of this collateral cytotoxicity and hypothesized that the non-specific cytotoxicity could be from contamination with αβ T cells. To test this hypothesis, we ran flow analysis of enriched Vδ2 T cells across multiple donors to determine the contamination level of ab T cells. We found that there is a donor-to-donor variability in the percentage of Vδ2 T enrichment after ZOL treatment. For example, in one donor ZOL treatment resulted in 90% pure Vδ2 T cells and less that 10% αβ T cells, but only 55% Vδ2 T cells and 35% αβ T cells in another donor. Even in a donor with 85% Vδ2 T, there were 5% CD3−CD56+ (NK cells) and 8.5% CD3+γδTCR− CD56+ cytotoxic cells (αβ T cells) which might contribute to the non-specific cytotoxicity towards non-SC.

Next, we asked if the enrichment of γδT cells is inversely correlated to the non-specific cytotoxicity towards non-SC. in fact, donors with high enrichment of γδT cells exhibited lower cytotoxicity towards non-SC compared to donors with weak enrichment of γδT cells. Conversely, donors with higher levels of CD8+ T cell contamination tended to have higher cytotoxicity towards non-SC.

Finally, we asked if depletion of αβ T cells after enrichment will improve the non-specific cytotoxicity towards non-SC. To this, we first depleted all γδTCR− cells from the donor with only 55% Vδ2 T cells and 35% αβ T cells using the Miltenyi biotech® γδ T cell isolation kit and isolated to further purify Vδ2 T cells. By this method we able to deplete αβT cell contamination in cells from this donor from 35% to 1.5% and further elevate the enrichment of γδT cells from 55% to 95%. We repeated this protocol in cells from other donors and similarly depleted αβ T cells and further enriched γδT cells. We then compared the cytotoxicity toward senescent or non-SC of either Vδ2 T cells enriched using our initial method or Vδ2 T cells depleted of αβ T cells as above. As anticipated, enriched and αβ-depleted Vδ2 cells are equally cytotoxic toward SC as Vδ2 cells enriched using our earlier protocol, but exert significantly less cytotoxicity towards non-SC.

### Example 4

### TCR and NKG2D Receptors are Required for the Vδ2 T Cell Mediated Killing of Senescent Cells

It has been reported that human γδT cells employ several distinct mechanisms to kill pathogens and tumor cells. γδT cells recognize pathogens based on a cell contact-dependent mechanism through the NK receptor NKG2D or through the γδ TCR The cytotoxicity of γδT cells can then be mediated through the perforin-granzyme pathway, the secretion of proinflammatory cytokines such as TNF-α and IFN-γ, or proapoptotic molecules such as FasL and TRAIL. Here we investigated whether NKG2D and γδ TCR are required for the cytotoxicity of γδT cells towards SC.

We assessed the mechanisms responsible for recognition of SC by Vδ2 T cells by individually blocking NKG2D or γδTCR receptors and coculturing them with doxorubicin treated senescent IMR90 cells. Although the cytotoxicity of Vδ2 T cells towards SC was significantly inhibited by either anti-γδTCR or anti-NKG2D neutralizing antibodies, Vδ2 T cells retained appreciable cytotoxicity even after blocking each receptor individually. We therefore asked if simultaneously blocking of γδTCR and NKG2D would further reduce the cytotoxicity Indeed, simultaneous blocking of NKG2D and γδTCR significantly reduced the cytotoxicity compared to blocking each receptor alone. This indicates that both NKG2D and TCR can participate in recognition and killing of SC by Vδ2 T cells, but the residual activity suggests some further mechanism of cytotoxicity is also involved.

### Example 5

### Mevalonate Pathway of Senescent Cells is Vital for the Cytotoxic Activity of Vδ2 T Cells

NKG2D recognizes target cells based on their surface expression of ligands such as MICA. Previous studies showed that SC express high levels of MICA on their surfaces (e.g., Sagiv et al, Againg, 8, 328-244, 2016). γδTCR additionally recognize targets based on sensing of phosphoantigens, which depends on the transmembrane proteins butyrophilin 3A1 (BTN3A1) and BTN2A1. Briefly, phosphoantigens such as IPP (an intermediate metabolite of mevalonate pathway) bind to the intracellular domain of BTN3A, leading to a conformational change in the extracellular domain that is recognized by γδTCR. We showed that SC have high levels of BNT3A on their surface. To determine the role of mevalonate pathway, we inhibited the rate-limiting enzyme 3-hydroxy-3-methylglutaryl-CoA reductase (HMGCR) with mevastain in SC and tested the effect on cytotoxicity by Vδ2 T cells. We found that mevalonate pathway inhibition in SC significantly reduced Vδ2 T cell-mediated cytotoxicity toward them. This indicates that of mevalonate pathway is required for SC destruction by Vδ2 T cells, consistent with a role for phosphoantigens such as IPP in SC immunosurveillance by this cell type.

### Example 6

### Therapeutic Removal of Senescent Cells

Studies show that removal of senescent cells increases health-span in progeroid mouse models, and transplantation of a relatively small number of senescent cells into previously healthy animals provokes multisystem dysfunction similar to what is seen in aged animals. Further, it was found that the correlation of senescent cell accumulation with disease extends to humans, and that senescent cell burden can be safely reduced in a clinical context.

Adoptive cell transfer (ACT) is the transfer of cells into a patient. The cells may have originated from the patient or from another individual. The cells are most commonly derived from the immune system with the goal of improving immune functionality and characteristics. In autologous cancer immunotherapy, T cells are extracted from the patient, genetically modified and cultured in vitro and returned to the same patient. Comparatively, allogeneic therapies involve cells isolated and expanded from a donor separate from the patient receiving the cells.

In this example, a patient (65-year-old male) visits a clinic and presents signs and symptoms of inflammation, atherosclerosis and hypertension. A healthcare provider suspects that the targeted removal of senescent cells in the patient will improve the patient's cardiovascular ailments.

In this example, gamma delta T cells are obtained from peripheral blood mononuclear cells (PBMCs) from ten volunteer donors. The γδT cells were isolated and enriched from human blood as described above using zoledronate and interleukin-2 (also see, e.g., Kondo, M. et al. J Vis Exp, 2008). Blood samples were diluted with 1× PBS and combined with Lymphocyte Separation Medium followed by density gradient centrifugation. The aliquot of PBMC was analyzed for the proportion of the γδT cells by flow cytometry. The remainder of PBMCs were cultured in RPMI complete media, 20% Fetal Bovine Serum (FBS) with 500 IU/ml human rIL-2 (recombinant Interleukin-2) and 5 uM Zoledronic Acid (Zol) for 24 hours. The cells were expanded with rIL-2 for ten days with fresh media changed every other day, before co-culturing with senescent or non-senescent IMR-90 fibroblasts.

The patient is administered a single dose (i.e., half of the enriched sample) of the gamma delta T cells intravenously. A second dose (i.e., the remainder) is administered after 30 days. The γδT cells selectively target the destruction of senescent cells in the patient. Further, the gamma delta T cells also slow the aging process and reduce signs of aging. Within two weeks of the second dose, the patient's blood pressure has improved. The healthcare provider continues to monitor levels of inflammation, atherosclerosis and hypertension.

### Example 7

### Gamma Delta Cells for Decreasing Senescent Cell Burden

Cellular senescence is a primary aging process and tumor suppressive mechanism characterized by irreversible growth arrest, apoptosis resistance, production of a senescence-associated secretory phenotype (SASP), mitochondrial dysfunction, and alterations in DNA and chromatin. In preclinical aging models, accumulation of senescent cells is associated with multiple chronic diseases and disorders, geriatric syndromes, multimorbidity, and accelerated aging phenotypes. In animals, genetic and pharmacologic reduction of senescent cell burden results in the prevention, delay, and/or alleviation of a variety of aging-related diseases and sequelae. Early clinical trials have thus far focused on safety and target engagement of senolytic agents that clear senescent cells. In this example, γδT cells are administered to a patient to clear senescent cells.

Senescent cell burden can be determined by markers that are present at increased levels in senescent cells. Besides p16 and p21, a number of other markers can be used to identify senescent cells, although sensitivity and specificity varies for each (see, e.g., Gasek et al, Nature Aging 1, 870-892, 2021). Applicants have identified LAMP-1 and TPP-1 as surface biomarkers on senescent cells. Also, morphologic features are suggestive of senescence in vitro and can be assessed using approaches such as brightfield microscopy. Compared to their counterparts, senescent cells have increased size and granularity, likely reflecting their altered metabolism and organelle homeostasis.

In this example, a patient (65-year-old female) visits a physicians' clinic and presents signs and symptoms of senescent cell burden. Specifically, the patient presents signs/symptoms of chronic inflammation, osteoarthritis and frailty. A healthcare provider suspects that the targeted removal of senescent cells in the patient will improve the patient's overall health. In addition to their internal homeostatic perturbations, senescent cells can induce an inflammatory state that provokes both local and systemic inflammation and tissue damage through their SAS.

In this example, gamma delta T cells are obtained from peripheral blood mononuclear cells (PBMCs) from ten volunteer donors and enriched as described above. The patient is administered a single dose (i.e., half of the enriched sample) of the gamma delta T cells intravenously. A second dose (i.e., the remainder) is administered after 30 days. The γδT cells selectively target the destruction of senescent cells in the patient. This reverses/slows the aging process. The healthcare provider continues to monitor the patient's inflammation and osteoarthritis. After the treatment, the patient expresses relief of pain from inflammation. She also feels stronger and more alert.

### Methods of Use

Embodiments include a method of treating an ailment (i.e., a senescence-associated disease or disorder) and/or slowing the aging process or reducing signs of aging. The method can include enriching and administering a solution of gamma delta T cells to a subject. Embodiments also include therapies for treating a senescence-associated disease or disorder and slowing the aging process. In one embodiment, a method includes administering to a pharmaceutical formulation containing a therapeutic agent that selectively kills senescent cells (i.e., selectively kills senescent cells over non-senescent cells or compared with non-senescent cells). A treatment regimen can include administering a pharmaceutical formulation for a time sufficient and in an amount sufficient to selectively kill senescent cells. The pharmaceutical formulation can include gamma delta T cells along with another senolytic agent.

The therapeutic method of the present specification can include the step of administering drug product (e.g., gamma delta T cells) at a pharmaceutically effective amount. The total daily dose should be determined through appropriate medical judgment by a physician and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In still another aspect, the present specification provides a use of the pharmaceutical composition including the same in the preparation of drugs for the prevention or treatment of a senescence-associated disease or disorder and/or slowing the aging process/reducing signs of aging.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the agent(s) disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and preferably is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose, or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the peptide is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

Given the teachings and guidance provided herein, those skilled in the art will understand that a formulation described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

Dosing can be single dosage or cumulative (i.e., serial dosing), and can be readily determined by one skilled in the art. For example, treatment of a senescence-associated disease or disorder can comprise a one-time administration of an effective dose of a pharmaceutical composition disclosed herein. Alternatively, treatment of a senescence-associated disease or disorder may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a therapeutic (e.g., solution of enriched γδT cells) disclosed herein is capable of reducing the signs/symptoms of a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects, a therapeutic is capable of reducing the number of signs/symptoms of a senescence-associated disease or disorder in an individual by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic disclosed herein is capable of reducing signs/symptoms in an individual suffering from a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms in an individual suffering from a senescence-associated disease or disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a therapeutic and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a therapeutic is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a therapeutic (e.g., γδT cell formulation) disclosed herein reduces signs/symptoms in an individual suffering from a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces signs/symptoms by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces signs/symptoms by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutically effective amount of a therapeutic disclosed herein reduces the aging process and/or reduces signs of aging in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces the aging process and/or reduces signs of aging by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces the aging process and/or reduces signs of aging by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutically effective amount of a therapeutic (e.g., enriched formulation of γδT cells) disclosed herein reduces senescent cell burden in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces the senescent cell burden by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces senescent cell burden by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In aspects, senescent cell burden is measured using biomarkers or comparing cell morphology. In aspects, the reduction in senescent cell burden slows/reverses signs of aging.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1. A method of treating an ailment, the method comprising administering a therapeutic amount of gamma delta T cells to a subject.

2. The method of claim 1, wherein the gamma delta T cells selectively target senescent cells for destruction.

3. The method of claim 1, wherein the gamma delta T cells target secondary senescent cells.

4. The method of claim 1, wherein the gamma delta T cells target primary senescent cells and secondary senescent cells.

5. The method of claim 1, wherein the ailment is a senescence-associated disease or disorder.

6. The method of claim 5, wherein the senescence-associated disease or disorder is one or more of atherosclerosis, osteoarthritis, osteoporosis, hypertension, arthritis, cataracts, cancer, Alzheimer's disease, chronic obstructive pulmonary disease (COPD) and idiopathic pulmonary fibrosis, hair graying, sarcopenia, adiposity, neurogenesis, fibrosis and glaucoma.

7. The method of claim 1, further comprising a step of modifying the gamma delta T cells.

8. The method of claim 7, wherein the step of modifying the gamma delta T cells comprises T-cell receptor (TCR) gene transfer or chimeric antigen receptor (CAR) expression.

9. The method of claim 1, wherein the method further comprises administration of a senolytic agent to the subject.

10. The method of claim 9, wherein the senolytic agent is selected from dasatinib, quercetin, fisetin and navitoclax.

11. The method of claim 1, further comprising a step of activating the gamma delta T cells.

12. The method of claim 11, wherein the gamma delta T cells are activated by zoledronate or adoptive transplantation.

13.-27. (canceled)

28. A method of treating senescence-associated disease or disorder, slowing the aging process and/or reducing signs of aging, the method comprising the steps of:
a) isolating and/or enriching gamma delta T cells, and
b) administering the gamma delta T cells to a subject.

29. The method of claim 28, wherein the senescence-associated disease or disorder is one or more of atherosclerosis, osteoarthritis, osteoporosis, hypertension, arthritis, cataracts, cancer, Alzheimer's disease, chronic obstructive pulmonary disease (COPD) and idiopathic pulmonary fibrosis, hair graying, sarcopenia, adiposity, neurogenesis, fibrosis and glaucoma.

30. The method of claim 28, further comprising a step of modifying the gamma delta T cells by T-cell receptor (TCR) gene transfer or chimeric antigen receptor (CAR) expression.

31. The method of claim 28, further comprising a step of administering a therapeutic amount of a senolytic agent to the subject.

32. The method of claim 31, wherein the senolytic agent is selected from dasatinib, quercetin, fisetin and navitoclax.

33. The method of claim 28, wherein the step of enriching and isolating gamma delta T cells comprises treating cells with zoledronate and/or interleukin-2.

34. The method of claim 28, wherein the step of enriching and isolating gamma delta T cells comprises a step of obtaining PBMCs from a plurality of human donors.

35. The method of claim 28, wherein the gamma delta T cells are administered to the subject intravenously.

36.-43. (canceled)
